(19) United States
(12) Patent Application Publication
(10) Pub. No.: US 2026/0272787 A1
(43) Pub. Date: Sep. 17, 2026

(54) METHOD OF IMPROVING STABILITY OF DENTIN BONDING INTERFACE USING PHOSPHORYLATED CHITOSAN

(71) Applicant: School and Hospital of Stomatology, Guangzhou Medical University, Guangzhou (CN)

(72) Inventors: Jie XIONG, Guangzhou City (CN); Ziliang ZHOU, Guangzhou City (CN); Yanyan YANG, Guangzhou City (CN); Lu HE, Guangzhou City (CN); Xin DENG, Guangzhou City (CN); Siyuan CHEN, Guangzhou City (CN)

(21) Appl. No.: 19/433,173

(22) Filed: Dec. 26, 2025

(30) Foreign Application Priority Data
Mar. 13, 2025 (CN) 2025102942871

(51) Int. Cl. A61K 6/54; A61K 6/60; A61K 6/838

(52) U.S. Cl. CPC A61K 6/54; A61K 6/60; A61K 6/838

(57) ABSTRACT

The present disclosure provides a method of improving stability of a dentin bonding interface using phosphorylated chitosan, belonging to the technical field of biomedicine. The method according to the present disclosure includes: applying a mixture of phosphorylated chitosan, 1-(3-dimethylaminopropyl)-3-ethylcarbodiimide (EDC), and N-hydroxysuccinimide (NHS) uniformly onto a dentin surface to form a covalently cross-linked network and induce in situ deposition of hydroxyapatite. The present disclosure achieves the following performance enhancements to the dentin bonding interface: the present disclosure increases the mechanical strength and enzymatic degradation resistance of dentin collagen fibers. The present disclosure imparts bacterial inhibition capability to the bonding interface, thereby preserving demineralized dentin collagen fibers and stabilizing the bonding interface. The present disclosure stabilizes the dentin collagen fibers, maintains a microenvironment suitable for remineralization, promotes the deposition of minerals such as calcium and phosphorus on the dentin surface, and also provides protection for the dentin against demineralization.

### CROSS-REFERENCE TO RELATED APPLICATIONS

This application claims priority to Chinese Patent Application No. 202510294287.1, filed on Mar. 13, 2025, which is incorporated herein by reference in its entirety.

### TECHNICAL FIELD

The present disclosure belongs to the technical field of biomedicine, and particularly relates to a method of improving stability of a dentin bonding interface using phosphorylated chitosan (PC).

### BACKGROUND

With the continuous development of dental materials, various types of dental restorative materials are widely used in clinical practice. The success of the dental restorative materials bonding to dental hard tissues is a crucial factor in determining their clinical application value. Dentin bonding is a complex physicochemical adhesion process. Although the immediate bond strength achieved by current bonding techniques can meet clinical requirements, the bond strength gradually declines over time as the restoration is subjected to the complex oral environment, leading to issues such as marginal microleakage and secondary caries, and ultimately resulting in bonding failure of the restoration. Therefore, improving the durability of dentin bonding is currently a significant issue for dentin bonding quality.

The durability of dentin bonding depends on the aging of the weakest link within the bonding interface, that is, the adhesive substrate. Within the bonding interface, due to the mismatch between the depth of dentin demineralization and the infiltration of adhesive/resin, and the hydrolysis of unpolymerized resin monomers and water-soluble molecules under the influence of various factors such as water and enzymes in the oral environment, the amount of exposed collagen fibers increases. These fibers then undergo degradation under the action of endogenous and exogenous collagenases, greatly reducing the micromechanical interlocking between the adhesive/resin and dentin. Consequently, protecting the exposed collagen fibers at the bonding interface is an important approach to improving the durability of dentin bonding.

In preliminary research for the present disclosure, chitosan nanoparticles (Csnp) of different molecular weights, under the action of the cross-linking agent 1-(3-dimethylaminopropyl)-3-ethylcarbodiimide (EDC), firmly bound to the surface of the triple-helical structure of collagen molecules, encapsulating the collagen at a molecular level to isolate collagen molecules from contact and adhesion by collagenases and bacteria (FIG. 1). This reduces the degradation of collagen fibers at the bonding interface and enhances the bonding interface's resistance to hydrolysis or enzymatic degradation. PC uses the chitosan molecule as its backbone, inheriting chitosan's superior properties such as biodegradability and biocompatibility. The amino groups, which are the reactive functional groups of chitosan serving as the molecular structural basis for the chemical cross-linking between EDC and collagen fibers, are also retained in this type of molecule. Simultaneously, due to the introduction of phosphate groups, PC possesses characteristics such as calcium ion chelation, dentin inductivity, and antibacterial properties. Mediated by the cross-linker EDC, PC forms a covalent bond with collagen fibers in a “zero-length” manner. The cross-linked PC is fixed onto the collagen fibers, increasing the stability of the collagen fibers and preventing their detachment or dissociation during subsequent procedures. Pretreating the dentin bonding interface in this manner has the effect of reinforcing dentin collagen fibers and providing resistance to enzymatic degradation. Under the influence of this pretreatment, the exposed collagen fibers in dentin gain more stable resistance to enzymatic degradation, thereby simultaneously creating conditions for gradual remineralization. This can guide the deposition of calcium and phosphate ions to re-embed the collagen fibers while improving the collagen fibers' resistance to enzymatic degradation, fundamentally protecting the dentin collagen fibers, which are the weakest link in the bonding interface, from aging due to external stimuli. This achieves “treating both the symptoms and the root cause” to maintain dentin bonding durability.

Existing research on protecting collagen fibers in demineralized dentin can be categorized into two types: The first type involves reinforcing the collagen fibers or inhibiting collagenase activity, to some extent reducing the amount of collagen fiber degradation or prolonging the time before degradation takes effect. This “treats the symptoms” of bonding interface aging caused by collagen exposure followed by enzymatic degradation, hydrolysis, etc. The second type aims to seal the exposed collagen, which is the weak link of the bonding interface, as much as possible (through sufficient infiltration of adhesive or redeposition of dentin minerals), thereby forming a stable bonding interface complex to isolate the complex from endogenous and exogenous stimuli, creating conditions where processes like enzymatic degradation and hydrolysis cannot occur, i.e., “treating the root cause” (FIG. 2). However, neither type of protection method alone can effectively and thoroughly solve the problem of bonding interface instability caused by collagen fiber degradation: “Treating the symptoms” alone means collagen fibers ultimately cannot escape degradation; “treating the root cause” alone is limited by the time required for mineralization and lacks the stable conditions necessary to complete mineralization.

### SUMMARY

The objective of the present disclosure is to develop a method that, through pretreatment of the dentin bonding interface, can inhibit dentin collagen degradation and stabilize the collagen while simultaneously promoting the orderly deposition of inorganic substances such as calcium and phosphorus to seal the collagen, thereby providing comprehensive protection for the collagen fibers and achieving the goals of stabilizing the bonding interface and improving bonding durability.

A first objective of the present disclosure is to provide a method of improving stability of a dentin bonding interface using PC, wherein the method includes:
applying a mixture of PC, EDC, and N-hydroxysuccinimide (NHS) uniformly onto a dentin surface to form a covalently cross-linked network and induce in situ deposition of hydroxyapatite.

Preferably, a phosphorus content of the PC is ≥5%.

Preferably, a concentration of the phosphorylated chitosan is from 0.5 g/L to 2.0 g/L; a concentration of the EDC is from 2.4 mol/L to 4 mol/L; and a concentration of the NHS is from 0.5 mol/L to 1.2 mol/L.

Preferably, a concentration of the PC is 1 g/L; a concentration of the EDC is 3.3 mol/L; and a concentration of the NHS is 0.8 mol/L.

Preferably, a method of preparing the PC includes: using a microwave-assisted method with 1,2,4-butanetricarboxylic acid as a phosphate source to modify a C6 hydroxyl group of chitosan via an esterification reaction, to prepare the PC with a phosphorus content of ≥5%.

A second objective of the present disclosure is to provide a composition for improving bonding between PC and demineralized dentin, including cross-linking agents EDC and NHS; wherein a molar concentration ratio of the EDC to the NHS is (2.4-4):(0.5-1.2) mol/L.

A third objective of the present disclosure is to provide an oral adhesive restorative material, including PC, EDC, and NHS; wherein a phosphorus content of the PC is ≥5%.

Preferably, a concentration of the PC is from 0.5 g/L to 2.0 g/L; a concentration of the EDC is from 2.4 mol/L to 4 mol/L; and a concentration of the NHS is from 0.5 mol/L to 1.2 mol/L.

A fourth objective of the present disclosure is to provide a use of the aforementioned composition for improving bonding between PC and demineralized dentin, or the aforementioned oral adhesive restorative material, in the preparation of a dental restorative material.

The advantageous technical effects of the present disclosure are the following performance enhancements to the dentin bonding interface:
1. The present disclosure increases the mechanical strength and enzymatic degradation resistance of dentin collagen fibers.
2. The present disclosure imparts bacterial inhibition capability to the bonding interface, thereby preserving demineralized dentin collagen fibers and stabilizing the bonding interface.
3. The present disclosure stabilizes the dentin collagen fibers, maintains a microenvironment suitable for remineralization, promotes the deposition of minerals such as calcium and phosphorus on the dentin surface, and also provides protection for the dentin against demineralization.
4. Through the synergistic effect of covalent crosslinking (anti-degradation) and phosphate group-guided mineralization (pro-regeneration), the present disclosure achieves “combined prevention and treatment”. For the first time in the field of dentin bonding, the present disclosure simultaneously addresses the three major challenges of microleakage, collagen degradation, and remineralization, improving restoration longevity by more than two times.

### BRIEF DESCRIPTION OF THE DRAWINGS

The present disclosure is further explained with reference to the accompanying drawings. However, the embodiments in the drawings do not constitute any limitation on the present disclosure. A person of ordinary skill in the art may derive other figures based on the following drawings without creative effort.

FIG. 1 is a molecular structural analysis of chitosan occupying the collagen-CAN binding site.

FIG. 2 is a schematic diagram illustrating the protection of demineralized dentin collagen by PC.

FIG. 3 is a schematic diagram of the preparation process for an extracted tooth.

FIG. 4 is a schematic diagram of EDC-crosslinked chitosan and human type I collagen.

FIG. 5 shows the characterization of demineralized dentin samples.

FIG. 6 shows the characterization of remineralized dentin samples.

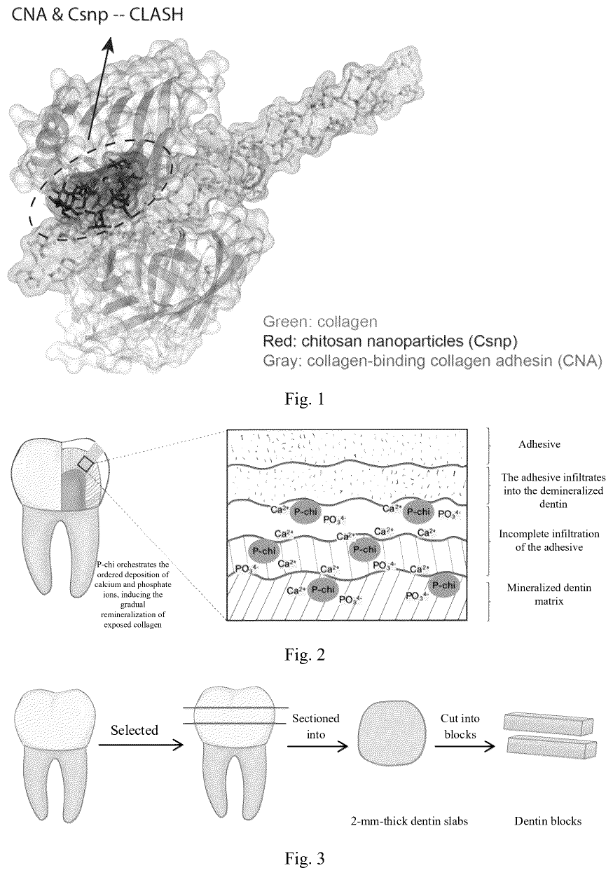

### DETAILED DESCRIPTION OF THE EMBODIMENTS

To better understand the present disclosure, further details are provided below in conjunction with specific embodiments. The terms used in the embodiments are intended to describe specific embodiments only and do not limit the scope of protection of the present disclosure.

It should be noted that the workflow described above is merely schematic and does not limit the scope of protection of the present disclosure. In practical applications, a person skilled in the art may select part or all of the described steps to achieve the objectives of the embodiments according to actual needs, with no limitations imposed herein.

### Definitions of Abbreviations in the Present Disclosure

PC: Phosphorylated Chitosan
EDC: 1-(3-Dimethylaminopropyl)-3-ethylcarbodiimide
NHS: N-Hydroxysuccinimide
M: mol/L
CDDM: Completely Demineralized Dentin Matrix
SEM: Scanning Electron Microscope
EDS: Energy Dispersive Spectrometer

### Embodiment 1

### 1. Preparation of Demineralized Dentin

Following approval from the ethics committee and informed patient consent, freshly extracted, caries-free, crack-free, and defect-free molars were collected. Immediately after extraction, the teeth were cleaned with a 3% hydrogen peroxide solution and stored in physiological saline at 4° C. The teeth were used within one month after extraction. For use, the teeth were embedded and fixed in a mold with embedding resin. Using a slow-speed cutting machine under water-cooling conditions, dentin slabs approximately 0.5 mm thick were sectioned perpendicular to the long axis of the tooth at the mid-coronal region, ensuring flat upper and lower surfaces (FIG. 3). The dentin slabs were then wet-polished with 600-grit silicon carbide sandpaper, followed by demineralization using a 10% phosphoric acid solution. The degree of demineralization was assessed by Fourier Transform Infrared Spectroscopy (FTIR). A reduction in the vibrational peak at 1004 cm⁻¹, representing the PO₄³⁻ group, indicated that the dentin slab had reached a partially demineralized state. The demineralized dentin collagen slabs were ultrasonically cleaned three times with deionized water to remove the phosphoric acid solution, then sectioned into dentin strips measuring 3 mm×3 mm×0.5 mm. The strips were stored at 4° C. for subsequent use.

### 2. Chemical Crosslinking Reaction

### (1) Crosslinking and Fixation of PC to Collagen Fibers on the Surface of Demineralized Dentin

The demineralized dentin strips were placed in 1 mL of a solution containing 3.3 M cross-linking agent EDC, 0.8 M NHS, and 1 g/L PC, and incubated at 4° C. for 24 hours. Crosslinking occurred between the carboxyl groups of the collagen fibers and the amino groups of PC, mediated by EDC.

The detailed crosslinking principle can be found in the article: “Zhou Z, Yang Y, He L, Wang J, Xiong J. Molecular docking reveals Chitosan nanoparticle protection mechanism for dentin against Collagen-binding bacteria. J Mater Sci Mater Med 2022, 33(5):43.”

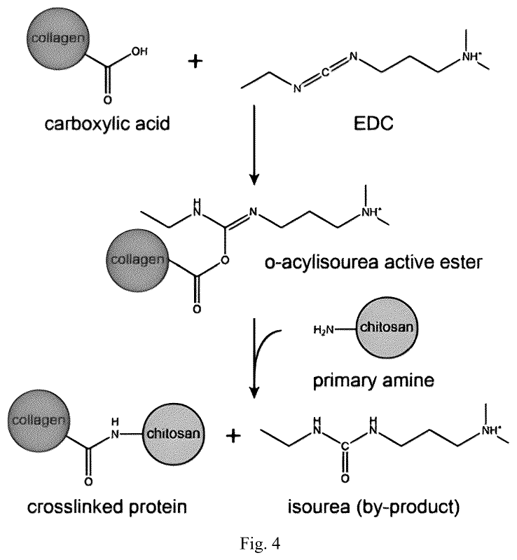

EDC first reacts with the carboxyl groups of collagen to form an intermediate containing O-acylisourea. This intermediate, stabilized by NHS, further reacts rapidly with the amino groups of PC to form amide bonds, thereby establishing chemical crosslinks between collagen and PC, while releasing water-soluble isourea by-products (FIG. 4).

(2) Crosslinking and Stabilization Among Collagen Fibers within the Demineralized Dentin

While treating the demineralized dentin using the method described above, crosslinking also occurs among spatially proximate carboxyl and amino groups of the collagen fibers within the demineralized dentin matrix, mediated by EDC. The crosslinking principle is similar to the method described above (FIG. 4).

The preparation of PC refers to the description in Chinese Patent Application CN 113185622 A, titled “high-phosphorus-content chitosan derivative, preparation thereof and application of the high-phosphorus-content chitosan derivative in loading nano zero-valent iron”. The phosphorus content in the PC of the present disclosure is ≥5%.

### Embodiment 2: Crosslinking and Effects of PC with CDDM Collagen Fibers

This embodiment examines the effects of covalent crosslinking of PC with collagen fibers on the surface of demineralized dentin on a resin adhesive applied to dentin, including surface composition, microstructure, and surface properties.

Specimen Preparation and Treatment: Following approval from the ethics committee and informed patient consent, 20 freshly extracted, caries-free, crack-free, and defect-free molars were collected (n=5). Using a slow-speed cutting machine under water-cooling conditions, dentin slabs approximately 0.5 mm thick were sectioned perpendicular to the long axis of the tooth at the mid-coronal region. The dentin slabs were then cut into sheet-shaped specimens measuring 3 mm×3 mm×0.5 mm, ensuring flat upper and lower surfaces (FIG. 3). The dentin surface intended for demineralization was then wet-polished sequentially with 400-grit, 800-grit, and 1200-grit silicon carbide sandpaper, ultrasonically agitated and cleaned for 5 minutes, and carefully examined under a stereomicroscope to ensure complete removal of any enamel on the dentin slab surface. Subsequently, nail polish was evenly applied to all surfaces except the surface intended for demineralization. The specimens were then immersed in a 10% phosphoric acid solution for demineralization for 24 hours, followed by rinsing with copious amounts of deionized water for 3 minutes, and oven-dried for 24 hours to obtain dry samples. The samples were processed according to the following experimental groups:
A. Control (Blank Control Group)
B. EDC Crosslinking Group (Negative Control Group)
C. PC Film-Forming Group
D. PC Crosslinking Group

For the film-forming group, the dentin specimen was placed in 100 μL of a PC solution and then placed in a desiccator until the water within the dentin completely evaporated. For the crosslinking group, the dentin slab was placed in 1 mL of a solution containing 3.3 M cross-linking agent EDC and 0.8 M NHS (with or without 1 g/L PC) and incubated at 4° C. for 24 hours.

a. Observation of Dentin Surface Micromorphology by SEM and Elemental Analysis by EDS

I. A total of 10 specimens from the treated groups (n=2 per group) were taken and rinsed with deionized water for 20 seconds.

II. The specimens were then fixed at room temperature with a solution of 3% glutaraldehyde and 0.1 mol/L phosphate-buffered saline (PBS) buffer (1:1, pH=7.2) for 4 hours, followed by three rinses with PBS. Subsequently, the specimens were dehydrated using a graded ethanol series (10%, 30%, 50%, 75%, 90%, and 100% ethanol solutions, three times each for 30 minutes per step).

III. The specimens were placed in 100% ethanol and dried using a critical point dryer with carbon dioxide. The dehydrated specimens were mounted on aluminum stubs and sputter-coated with a 20 nm layer of gold/palladium (60 Au: 40 Pd, Hummer X Sputter Coater, Anatech USA, Union City, CA, USA).

IV. The specimens were examined under an SEM at an accelerating voltage of 5 kV and a working distance of 10-12 mm to observe the structure of the collagen fibers on the dentin surface and the distribution of PC for each group. Eight images were captured for each specimen.

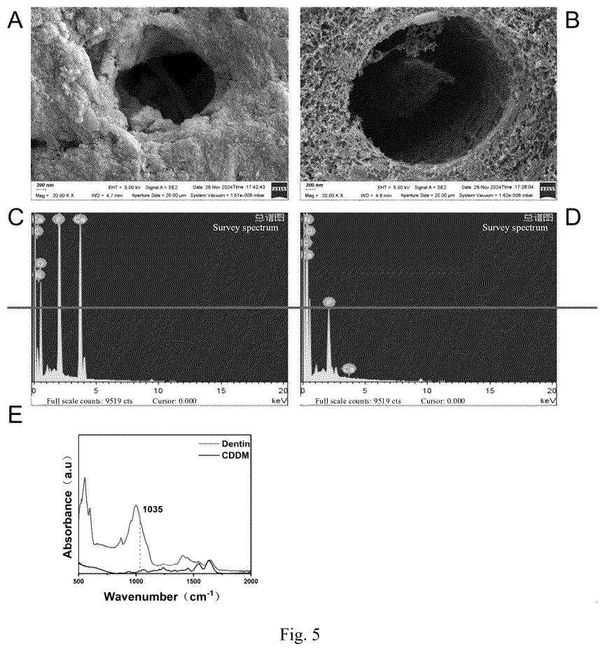

The demineralization status of the samples was observed under SEM at cross-sections of the dentinal tubules. The dentin slices from the control group appeared relatively flat with no distinct collagen fiber structure visible (A in FIG. 5). In contrast, the CDDM group exhibited a large number of collagen fibers, and the diameter of the dentinal tubules increased significantly (B in FIG. 5), indicating effective demineralization. EDS results showed that the phosphorus (P) and calcium (Ca) content in the CDDM group was significantly lower than that in the control group, indicating a substantial reduction in the mineral content of the samples (C, D in FIG. 5).

b. FTIR Analysis

FTIR spectra were obtained using the Attenuated Total Reflection (ATR) method in transmission mode on an infrared spectrometer (Shimadzu 8400S, Tokyo, Japan). The specific procedure was as follows: A total of 10 specimens from the treated groups (n=2 per group) were taken and rinsed with deionized water for 20 seconds. The specimens were dried and pressed into thin sheets. The specimens were then placed in the infrared spectrometer, and their infrared spectra were acquired using the infrared spectrometer in reflection mode. The wavenumber range was set from 400 to 4000 cm⁻¹ with a resolution of 16 cm⁻¹, and 100 scans were accumulated.

FTIR analysis results indicated that the infrared spectrum of the CDDM group displayed characteristic peaks of collagen. Compared to the non-demineralized dentin of the control group, the intensity of the P—O peak at 1035 cm⁻¹ was significantly reduced in the CDDM group, demonstrating that the CDDM group had achieved the intended effect of complete demineralization (E in FIG. 5).

### Embodiment 3: Collagenase Resistance Test

Preparation of Type I Collagenase Solution: A Dulbecco's Phosphate Buffered Saline (DPBS) buffer with pH=7.4 was purchased. Then, 50 mg of Type I collagenase with a specific activity of ≥125 CDU/mg was added to 100 mL of the DPBS buffer. The pH was adjusted to 7.4 and the solution was stirred until homogeneous.

a. Dry Weight Loss Experiment

Four demineralized dentin collagen strips from each group were taken and transferred to individual polypropylene centrifuge tubes. The strips were rinsed three times with deionized water and dried to a constant weight in a vacuum desiccator. Their mass was weighed using an analytical balance and recorded as m1, accurate to 0.01 mg. Subsequently, the dentin collagen strips were placed in 2 mL of the Type I collagenase solution for enzymatic digestion. Digestion was performed on a shaker in a 37° C. water bath for 24 hours. The remaining dentin collagen strips were removed, dried to a constant weight in a vacuum desiccator, and their remaining collagen mass was weighed using an analytical balance and recorded as m2.

The calculation formula is:

$$Dry Weight Loss = ( m 1 - m 2 ) / m 1 \times 100 \% .$$

TABLE 1
Collagenase Degradation Dry Weight Test

| | | | | PC |
|---|---|---|---|---|
| Measurement of Weight | Blank | PC Film- | EDC | Cross- |
| Before/After Digestion | Control | Forming | Control | linking |
| and Difference (mg) | Group | Group | Group | Group |
| After 1 day of collagenase | 0.514 | 0.066 | 0.439 | 0.042 |
| solution treatment | | | | |
| After 2 days of collagenase | 1.435 | 0.057 | 1.143 | 0.040 |
| solution treatment | | | | |

The collagenase degradation test was performed on the demineralized dentin of each group, and the mass loss of the dentin collagen strips after the reaction was measured and calculated. The free amino acid content detected in the PC crosslinking group was extremely low both after 1 day and 2 days of enzymatic digestion, measuring 0.042 mg and 0.040 mg, respectively. In contrast, the dry weight loss for the control PC film-forming group was 0.439 mg and 1.143 mg (Table 1). The results indicate that crosslinking PC with demineralized dentin can inhibit the enzymatic degradation of collagen fibers.

b. Amino Acid Release Experiment

From the polypropylene centrifuge tubes from which the dentin collagen strips had been removed, 0.5 mL of supernatant was taken for detection using an amino acid assay kit. Experimental steps were performed according to the kit's instructions for use. A microplate reader was used to measure the absorbance of the solution at a wavelength of 560 nm. The amount of amino acid released was calculated according to the kit's formula to evaluate the degradation of dentin collagen.

TABLE 2
Collagenase Degradation Amino Acid Release Test

| | | | | PC |
|---|---|---|---|---|
| Released Amino Acid | Blank | PC Film- | EDC | Cross- |
| Concentration | Control | Forming | Control | linking |
| (μmol/L · mg) | Group | Group | Group | Group |
| After 1 day of | 6.83 ± | 5.57 ± | 0.21 ± | 0.01 ± |
| enzymatic digestion | 0.16 | 0.22 | 0.27 | 0.23 |
| After 2 days of | 16.44 ± | 13.02 ± | 0.49 ± | −0.03 ± |
| enzymatic digestion | 0.29 | 0.44 | 0.21 | 0.34 |

The collagenase degradation test was performed on the demineralized dentin of each group, and the free amino acid content after the reaction was detected. The free amino acid content detected in the PC crosslinking group was extremely low both after 1 day and 2 days of enzymatic digestion, measuring 0.01±0.23 μmol/L·mg and −0.03±0.34 μmol/L·mg, respectively. In contrast, the free amino acid content detected in the control PC film-forming group was 5.57±0.22 μmol/L·mg and 13.02±0.44 μmol/L·mg, close to that of the blank control group, showing a statistically significant difference (p<0.05, Table 2). The results indicate that crosslinking PC with demineralized dentin can greatly protect its collagen fibers from degradation.

### Embodiment 4: PC Promotes Remineralization on the Surface of Demineralized Dentin

Preparation of Remineralization Solution: An artificial body fluid containing 1.7 mM calcium chloride, 9.5 mM disodium hydrogen phosphate, 125 mM sodium chloride, and 2.7 mg/ml sodium polyaspartate was prepared using Tris buffer at pH=7.4. The pH was adjusted to 7.4 using sodium hydroxide.

PC Solution Preparation: Prepared 24 hours before the experiment using MES (0.5 M, pH 5.0) as the solvent and stored at −20° C.

Remineralization Treatment of Demineralized Dentin Specimens: Specimens were immersed in the remineralization artificial fluid at 37° C. (30 mL per specimen), with the solution changed every 24 hours. The specimens were stored in the mineralization solution for 7 days and 14 days. After 7 days and 14 days, they were rinsed with deionized water for 3 minutes before detection.

a. Observation of Surface Micromorphology by SEM

I. A total of 8 specimens from the treated groups (n=2 per group) were taken and rinsed with deionized water for 20 seconds.

II. The specimens were then fixed at room temperature with a solution of 3% glutaraldehyde and 0.1 mol/L phosphate-buffered saline (PBS) buffer (1:1, pH=7.2) for 4 hours, followed by three rinses with PBS. Subsequently, the specimens were dehydrated using a graded ethanol series (10%, 30%, 50%, 75%, 90%, and 100% ethanol solutions, three times each for 30 minutes per step).

III. The specimens were placed in 100% ethanol and dried using a critical point dryer with carbon dioxide. The dehydrated specimens were mounted on aluminum stubs and sputter-coated with a 20 nm layer of gold/palladium (60 Au: 40 Pd, Hummer X Sputter Coater, Anatech USA, Union City, CA, USA).

IV. The specimens were examined under an SEM at an accelerating voltage of 5 kV and a working distance of 10-12 mm to observe the structure of the collagen fibers on the dentin surface and the distribution of PC for each group. Eight images were captured for each specimen.

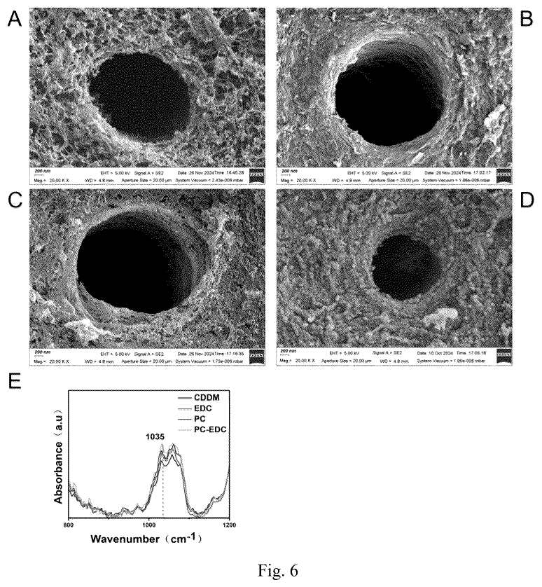

The remineralization status was observed from SEM images of the cross-sections of dentinal tubules from each group. Distinct mineralization particles were clearly observable on the surface of the collagen fibers in the PC crosslinking group, accompanied by thickening of the dentinal tubule walls and a reduction in tubule diameter (D in FIG. 6). In contrast, the other groups still exhibited visible collagen fiber structures (A, B, C in FIG. 6).

b. Detection by Fourier Transform Infrared Spectrometer (ATR-FTIR)

Qualitative analysis of the degree of demineralization on the dentin surface was determined by ATR-FTIR.

I. Ten teeth from each treatment group (n=2) were taken and rinsed with deionized water for 20 seconds.

II. The ATR-FTIR spectra of the dentin specimens were collected in reflection mode using an infrared spectrometer (SHIMADZU, 8400S, Japan) over the 400-4000 cm⁻¹ range, with a resolution of 16 cm⁻¹ and 10 scans. Each specimen was scanned at three different locations to analyze the content of mineral elements such as calcium and phosphorus on the specimen surface under different treatments.

The intensity of the P—O peak at 1035 cm⁻¹ was significantly increased in the PC crosslinking group, indicating a higher degree of remineralization in the demineralized dentin of the PC crosslinking group (E in FIG. 6).

### Embodiment 5: Scale-up Experiment with Different Concentrations

To further investigate the effect of different concentrations on remineralization, the following scale-up experiment was conducted.

The experiment was performed using the method of Embodiment 1, with the distinction that the concentrations of the cross-linking agent EDC, NHS, and PC in the chemical crosslinking reaction were as shown in Table 3 below. Then, the dry weight loss experiment and the hydroxyproline release experiment were conducted according to the method of Embodiment 3. The results are shown in Table 4.

TABLE 3
Different Concentration Groups for Scale-up Experiments

| | EDC | NHS | PC |
|---|---|---|---|
| | Concentration | Concentration | Concentration |
| Serial Number | (mol/L) | (mol/L) | (g/L) |
| Scale-up Experiment 1 | 2.0 | 0.2 | 0.2 |
| Scale-up Experiment 2 | 2.4 | 0.6 | 0.5 |
| Scale-up Experiment 3 | 4.0 | 1.2 | 2 |

TABLE 4
Results of Scale-up Experiments

| Measurement of Weight | Scale-up | Scale-up | Scale-up |
|---|---|---|---|
| Before/After Digestion | Experiment | Experiment | Experiment |
| and Difference (mg) | 1 | 2 | 3 |
| After 1 days of collagenase | 0.406 | 0.072 | 0.040 |
| solution treatment | | | |
| After 2 days of collagenase | 0.944 | 0.061 | 0.039 |
| solution treatment | | | |
| Released Amino Acid | Scale-up | Scale-up | Scale-up |
| Concentration | Experiment | Experiment | Experiment |
| (μmol/L · mg) | 1 | 2 | 3 |
| After 1 days of enzymatic | 4.18 ± 0.26 | 0.56 ± 0.04 | 0.56 ± 0.05 |
| digestion | | | |
| After 3 days of enzymatic | 6.23 ± 0.36 | 0.66 ± 0.05 | 0.62 ± 0.06 |
| digestion | | | |

As can be seen from the results in Table 4, when the PC concentration is 0.2 g/L, the EDC concentration is 2.0 mol/L, and the NHS concentration is 0.2 mol/L, the excessively low concentration of PC leads to insufficient mineralization induction capability. PC serves as the “template” for calcium and phosphate deposition. At too low a concentration, PC cannot adequately cover the collagen fiber surface, leaving residual collagen proteins susceptible to degradation by matrix metalloproteinases (MMPs), resulting in insufficient long-term stability.

When the NHS concentration is too low, the intermediate is prone to hydrolysis and deactivation, leading to an overly short window for the crosslinking reaction. Furthermore, EDC may preferentially react with free carboxylic acids in the solution (such as from PC or collagen hydrolysis products) rather than the target sites on the collagen fibers, resulting in ineffective crosslinking.

An excessively low EDC concentration leads to low efficiency in converting carboxyl groups to active esters (O-acylisourea), resulting in insufficient crosslink density between collagen molecules. Insufficiently crosslinked collagen cannot provide stable anchoring sites for PC, leading to decoupling between the mineralization layer and the collagen network. These factors collectively lead to a low degree of inhibition of the enzymatic degradation reaction of collagen fibers.

Conversely, if the PC concentration is too high, a locally excessive phosphate group concentration may occur, potentially forming disordered mineralization structures (such as coarse or unevenly distributed hydroxyapatite crystallites), which could instead reduce the mechanical strength of dentin. Excessively high PC concentration may also cover the active sites (such as carboxyl and amino groups) on the collagen fiber surface, hindering the EDC-mediated crosslinking reaction and leading to reduced crosslink density and decreased stability of the bonding interface. Additionally, high concentrations of PC may cause local pH fluctuations or cytotoxicity, inhibiting odontoblast activity and affecting pulp tissue health.

Excessively high EDC concentration leads to over-activation of the carboxyl groups on collagen fibers, causing excessively high intermolecular crosslink density. This increases the rigidity of the collagen network while reducing its toughness, making it prone to brittle fracture under stress. EDC is prone to hydrolysis, forming inactive urea derivatives. At high concentrations, side reactions accelerate, not only wasting reagents but also potentially generating impurities that interfere with the crosslinking process. EDC itself has cytotoxicity. Concentrations exceeding standards may remain within dentinal tubules, and long-term release could damage surrounding tissues (e.g., dental pulp).

Excessively high NHS concentration excessively prolongs the lifetime of the intermediate, making the crosslinking reaction time uncontrollable and potentially causing non-specific crosslinking (e.g., self-polymerization of PC molecules). High NHS concentration increases the ionic strength of the solution, reducing the penetration ability of PC and EDC into the depths of the dentinal tubules, resulting in only surface crosslinking without internal repair.

Residual clearance difficulty: When NHS is in excess, it may be difficult to remove completely through subsequent rinsing. Residual succinimide groups may trigger inflammatory reactions.

Therefore, for safety and mechanical performance considerations, the present disclosure did not conduct experiments with excessively high concentrations.

Furthermore, technical details not exhaustively described in this embodiment may be implemented by referring to the parameter operation methods provided in any embodiment of the present disclosure, and are not reiterated here.

It should be noted that in this document, the term “comprises,” “comprising,” or any other variation thereof is intended to cover a non-exclusive inclusion, such that a process, method, article, or system that comprises a list of elements does not include only those elements but may include other elements not expressly listed or inherent to such process, method, article, or system. An element preceded by “comprises a . . . ” does not, without more constraints, preclude the existence of additional identical elements in the process, method, article, or system that comprises the element.

The serial numbers of the above-described embodiments of the present disclosure are for description only and do not represent the superiority or inferiority of the embodiments.

The foregoing descriptions are merely preferred embodiments of the present disclosure and are not intended to limit the patent scope of the present disclosure. Any equivalent structural or process transformations made based on the description and drawings of the present disclosure, or direct or indirect applications in other related technical fields, shall similarly be included within the patent protection scope of the present disclosure.

What is claimed is:

1. A method of improving stability of a dentin bonding interface using phosphorylated chitosan, comprising:
applying a mixture of phosphorylated chitosan, 1-(3-dimethylaminopropyl)-3-ethylcarbodiimide (EDC), and N-hydroxysuccinimide (NHS) uniformly onto a dentin surface to form a covalently cross-linked network and induce in situ deposition of hydroxyapatite.

2. The method of improving stability of the dentin bonding interface using phosphorylated chitosan according to claim 1, wherein a phosphorus content of the phosphorylated chitosan is ≥5%.

3. The method of improving stability of the dentin bonding interface using phosphorylated chitosan according to claim 1, wherein a concentration of the phosphorylated chitosan is from 0.5 g/L to 2.0 g/L; a concentration of the 1-(3-dimethylaminopropyl)-3-ethylcarbodiimide (EDC) is from 2.4 mol/L to 4 mol/L; and a concentration of the N-hydroxysuccinimide (NHS) is from 0.5 mol/L to 1.2 mol/L.

4. The method of improving stability of the dentin bonding interface using phosphorylated chitosan according to claim 1, wherein a concentration of the phosphorylated chitosan is 1 g/L; a concentration of the 1-(3-dimethylaminopropyl)-3-ethylcarbodiimide (EDC) is 3.3 mol/L; and a concentration of the N-hydroxysuccinimide (NHS) is 0.8 mol/L.

5. The method of improving stability of the dentin bonding interface using phosphorylated chitosan according to claim 1, wherein a method of preparing the phosphorylated chitosan comprises: using a microwave-assisted method with 1,2,4-butanetricarboxylic acid as a phosphate source to modify a C6 hydroxyl group of chitosan via an esterification reaction, to prepare the phosphorylated chitosan with a phosphorus content of ≥5%.

6. A composition for cross-linking demineralized dentin with phosphorylated chitosan to improve stability of a dentin bonding interface, comprising cross-linking N-agents 1-(3-dimethylaminopropyl)-3-ethylcarbodiimide (EDC) and hydroxysuccinimide (NHS); wherein a molar concentration ratio of the EDC to the NHS is (2.4-4): (0.5-1.2).

7. An oral adhesive restorative material, comprising phosphorylated chitosan, 1-(3-dimethylaminopropyl)-3-ethylcarbodiimide (EDC), and N-hydroxysuccinimide (NHS); wherein a phosphorus content of the phosphorylated chitosan is ≥5%.

8. The oral adhesive restorative material according to claim 7, wherein a concentration of the phosphorylated chitosan is from 0.5 g/L to 2.0 g/L; a concentration of the 1-(3-dimethylaminopropyl)-3-ethylcarbodiimide (EDC) is from 2.4 mol/L to 4 mol/L; and a concentration of the N-hydroxysuccinimide (NHS) is from 0.5 mol/L to 1.2 mol/L.

9. Use of a composition for improving bonding between phosphorylated chitosan and demineralized dentin, or an oral adhesive restorative material, in the preparation of a dental restorative material; the composition for improving bonding between phosphorylated chitosan comprises cross-linking agents 1-(3-dimethylaminopropyl)-3-ethylcarbodiimide (EDC) and N-hydroxysuccinimide (NHS); wherein a molar concentration ratio of the EDC to the NHS is (2.4-4): (0.5-1.2); the oral adhesive restorative material, in the preparation of a dental restorative material comprises phosphorylated chitosan, 1-(3-dimethylaminopropyl)-3-ethylcarbodiimide (EDC), and N-hydroxysuccinimide (NHS); wherein a phosphorus content of the phosphorylated chitosan is ≥5%.
